(19) United States
(12) Patent Application Publication
(10) Pub. No.: US 2023/0190137 A1
(43) Pub. Date: Jun. 22, 2023

(54) INFORMATION PROCESSING SYSTEM, RECORDING MEDIUM, AND INFORMATION PROCESSING METHOD

(71) Applicant: SONY CORPORATION, TOKYO (JP)

(72) Inventor: AKIHIRO KOMORI, TOKYO (JP)

(21) Appl. No.: 16/314,699

(22) PCT Filed: Apr. 14, 2017
(86) PCT No.: PCT/JP2017/015348

§ 371 Date: Oct. 17, 2019

(30) Foreign Application Priority Data
Jul. 14, 2016 (JP) 2016-139779

(51) Int. Cl. A61B 5/11; A63F 13/825; G10L 25/63; G10L 25/66; A61B 5/00

(52) U.S. Cl. CPC A61B 5/1118; A61B 5/7264; A63F 13/825; G10L 25/63; G10L 25/66

(57) ABSTRACT

To provide a mechanism capable of suppressing long-term progress of a decline.

An information processing system including: an acquisition unit configured to acquire user-related information concerning a user; a learning unit configured to learn a normal state of the user on the basis of the user-related information; and an output control unit configured to, when an abnormal state of the user is detected by referring the acquired user-related information to the learned normal state, exert control so as to provide a task for suppressing the detected abnormal state.

### TECHNICAL FIELD

The present disclosure relates to an information processing system, a recording medium, and an information processing method.

### BACKGROUND ART

In recent years, people have come to live longer because of improvement in dietary habits, advancement in medical technologies, and the like. Thus, development of technologies for supporting a healthful life of the elderly is being demanded. As one of such technologies, a technology for promoting communication of the elderly, for example, can be considered.

For example, Patent Literatures 1 and 2 below disclose technologies concerning virtual pets that make communication with users. Patent Literature 1 below discloses a technology in which, in a system that breeds a virtual pet via a network, the pet sends a message such as “I miss you because you don’t come to see me these days” in accordance with an access history from a user. Patent Literature 2 below discloses a technology for, concerning a virtual pet to be grown in accordance with an activity of a user, utilizing changes in growing state of the virtual pet to give an advice to increase the moving speed so as to increase the amount of exercise of the user or the like, and perform health management of the user.

### CITATION LIST

### Patent Literature

Patent Literature 1: JP H10-328416A
Patent Literature2: JP 2010-214009A

### DISCLOSURE OF INVENTION

### Technical Problem

Here, people gradually decline in physical capacity and intellectual capacity with age for a long time. If progress of a decline due to aging is left, there is a danger such as stumbling on a step to be injured or leaving a gas fire on to cause a fire. Such a danger is urgent particularly for elderly people living alone. This is because, in the case of an elderly person living with his/her family, there are some cases where the family become aware of progress of a decline due to aging and can give appropriate assistance. However, considering that there are some cases where it is difficult to obtain assistance from the family, such a danger cannot be ignored also for an elderly person living with his/her family. Besides aging, the same also applies to the case where a decline is progressing in a long term because of a disease, lack of exercise, or the like, for example.

In this respect, with technologies such as those disclosed in Patent Literatures above, it is difficult to suppress long-term progress of a decline even if it is possible to improve a user’s short-term activity itself such as lack of access or a small amount of exercise. If long-term progress of a decline is suppressed, the occurrence of a danger resulting from that is also prevented. Therefore, the present disclosure proposes a mechanism capable of suppressing long-term progress of a decline.

### Solution to Problem

According to the present disclosure, there is provided an information processing system including: an acquisition unit configured to acquire user-related information concerning a user; a learning unit configured to learn a normal state of the user on the basis of the user-related information; and an output control unit configured to, when an abnormal state of the user is detected by referring the acquired user-related information to the learned normal state, exert control so as to provide a task for suppressing the detected abnormal state.

In addition, according to the present disclosure, there is provided a recording medium having recorded thereon a program for causing a computer to function as: an acquisition unit configured to acquire user-related information concerning a user; a learning unit configured to learn a normal state of the user on the basis of the user-related information; and an output control unit configured to, when an abnormal state of the user is detected by referring the acquired user-related information to the learned normal state, exert control so as to provide a task for suppressing the detected abnormal state.

In addition, according to the present disclosure, there is provided an information processing method including: acquiring user-related information concerning a user; learning a normal state of the user on the basis of the user-related information; and when an abnormal state of the user is detected by referring the acquired user-related information to the learned normal state, exerting control by a processor so as to provide a task for suppressing the detected abnormal state.

### Advantageous Effects of Invention

According to the present disclosure as described above, a mechanism capable of suppressing long-term progress of a decline is provided. Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a diagram for describing a concept of an information processing system according to the present embodiment.

FIG. 2 is a diagram for describing an overview of the information processing system according to the present embodiment.

FIG. 3 is a block diagram showing an example of a logical configuration of a terminal device according to the present embodiment.

FIG. 4 is a diagram for describing a flow of processing in CPU of the terminal device according to the present embodiment.

FIG. 5 is a flowchart showing an example of a flow of prior learning processing performed by the terminal device according to the present embodiment.

FIG. 6 is a flowchart showing an example of a flow of prior learning processing performed by the terminal device according to the present embodiment.

FIG. 7 is a flowchart showing an example of a flow of prior learning processing performed by the terminal device according to the present embodiment.

FIG. 8 is a flowchart showing an example of a flow of prior learning processing performed by the terminal device according to the present embodiment.

FIG. 9 is a flowchart showing an example of a flow of processing of detecting an abnormal state performed by the terminal device according to the present embodiment.

FIG. 10 is a flowchart showing an example of a flow of processing of detecting an abnormal state performed by the terminal device according to the present embodiment.

FIG. 11 is a flowchart showing an example of a flow of processing of detecting an abnormal state performed by the terminal device according to the present embodiment.

FIG. 12 is a diagram for describing an example of UI for a task provided by the terminal device according to the present embodiment.

FIG. 13 is a diagram for describing an example of UI for a task provided by the terminal device according to the present embodiment.

FIG. 14 is a diagram for describing an example of UI for a task provided by the terminal device according to the present embodiment.

FIG. 15 is a flowchart showing an example of a flow of processing of notifying a third party performed by the terminal device according to the present embodiment.

FIG. 16 is a diagram for describing an example of UI for a task provided by the terminal device according to the present embodiment.

FIG. 17 is a flowchart showing an example of a flow of processing of notifying a third party performed by the terminal device according to the present embodiment.

FIG. 18 is a diagram for describing an example of information of which a third party is to be notified by the terminal device according to the present embodiment.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that description shall be provided in the following order.
1. Overview
2. Configuration example
3. Technical characteristics
3.1. User-related information
3.2. Prior learning
3.3. Task provision
4. Conclusion

### 1. Overview

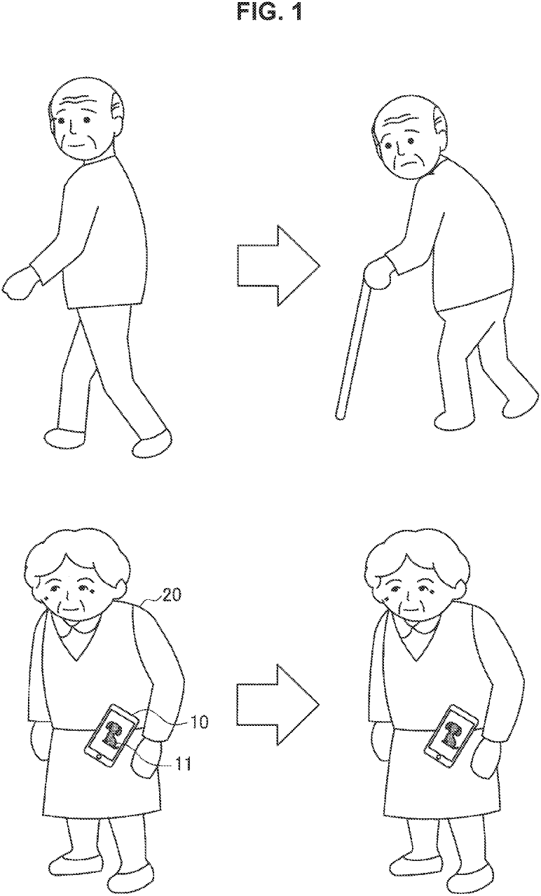

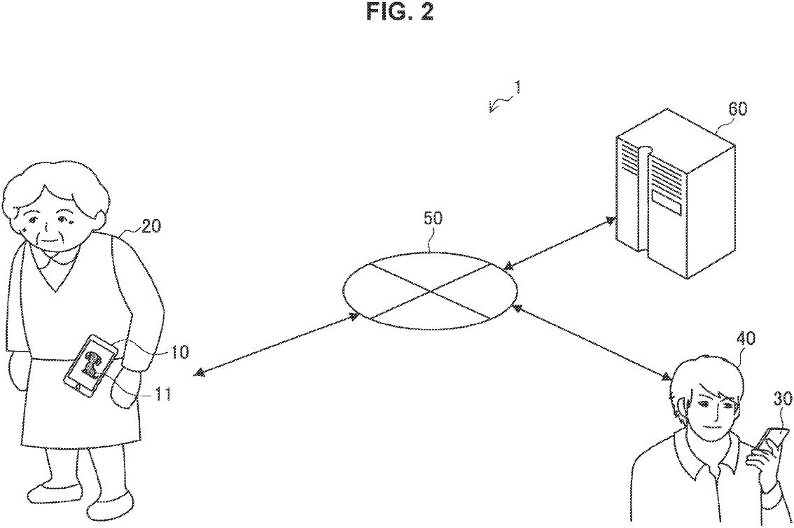

First, an overview of an information processing system according to an embodiment of the present disclosure will be described with reference to FIG. 1 and FIG. 2.

FIG. 1 is a diagram for describing a concept of the information processing system according to the present embodiment. As shown in FIG. 1, people experience a decline such as stooping with aging to need a stick. On the other hand, progress of a decline due to aging of a user 20 living with a terminal device 10 according to the present embodiment is suppressed to take a longer time than usual until the user 20 comes to stoop to need a stick, for example. That is, the information processing system according to the present embodiment is capable of suppressing progress of a decline due to aging. Note that, in the present specification, aging merely refers to getting older, and shall not include the concept of a decline due to aging.

Here, in the present specification, the functions of the terminal device 10 shall be provided via a virtual pet (that is, virtual organism) 11 operating on the terminal device 10. That is, the user 20 performs an interaction with the virtual pet 11 to be provided with a service for suppressing progress of a decline due to aging. Of course, the functions of the terminal device 10 may be provided without intervention of the virtual pet 11, or conversely, the terminal device 10 may be achieved as a device dedicated for a pet type robot or the like.

FIG. 2 is a diagram for describing an overview of the information processing system according to the present embodiment. As shown in FIG. 1, an information processing system 1 includes the terminal device 10, a terminal device 30, and a server 60.

The terminal device 10 is a device having the function of observing the user 20 and transmitting information concerning the user 20 to a remote place. The terminal device 10 acquires the information concerning the user 20 with a sensor or the like to provide a service based on the acquired information for the user 20 or to transmit the acquired information to the terminal device 30 of a third party 40 at a remote place via a network 50. For example, the user 20 is an elderly person living alone, and the third party 40 is the family of the user 20. Such a function enables the family 40 to confirm that the user 20 is healthy or that a decline due to aging is progressing, for example, from a remote place. Note that, as described above, such a function is provided via the virtual pet 11. For example, the terminal device 10 is implemented by a smartphone, a tablet terminal, an activity meter, or the like.

The terminal device 30 is a device that receives information concerning the user 20 from the terminal device 10, and outputs the received information to the family 40. For example, the terminal device 30 is implemented by a smartphone, tablet terminal, personal computer (PC), or the like.

The server 60 is provided on a cloud, for example, and manages the terminal device 10 included in the information processing system 1. For example, in order to assist in the operation of the terminal device 10, the server 60 makes an inquiry to a specialized institution such as a doctor, access to a database concerning the health, accumulation of necessary information, and the like.

The network 50 is a wired or wireless transmission path for information transmitted from a device connected to the network 50. The network 50 may include, for example, a local area network (LAN), a wireless LAN, Bluetooth (registered trademark), a long term evolution (LTE) network, and the like.

### 2. Configuration Example

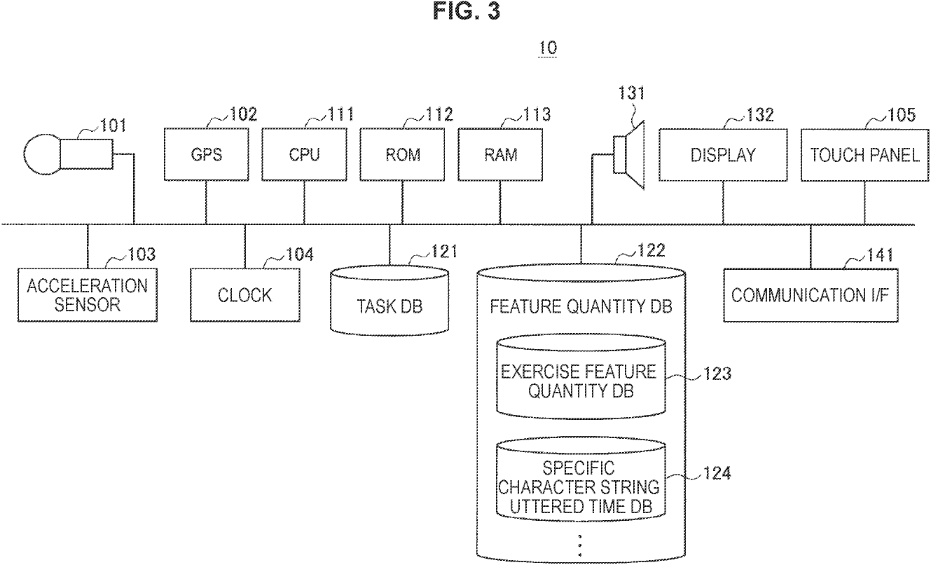

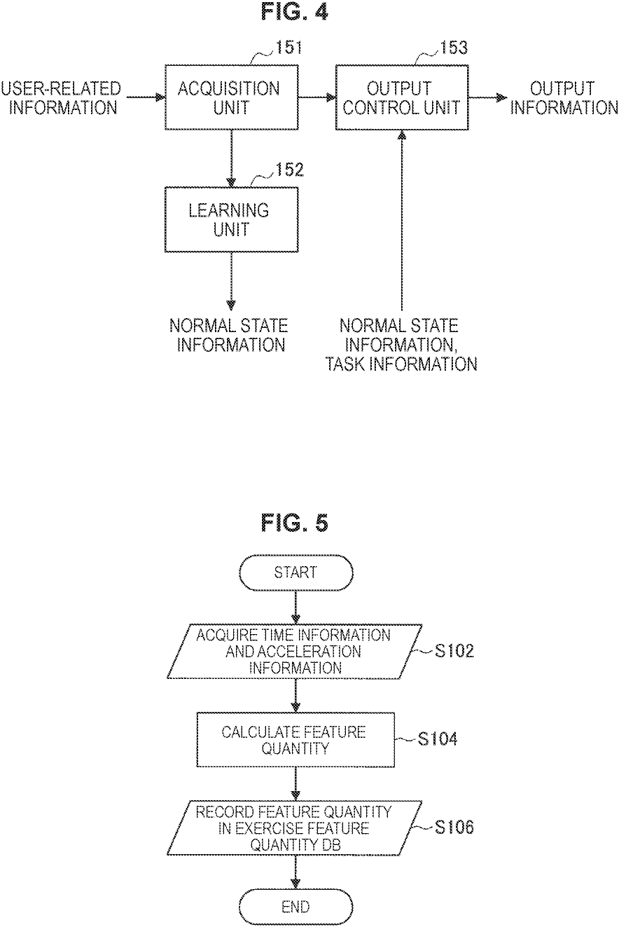

The overview of the information processing system 1 according to the present embodiment has been described above. Subsequently, a configuration example of the terminal device 10 according to the present embodiment will be described with reference to FIG. 3 and FIG. 4.

FIG. 3 is a block diagram showing an example of a logical configuration of the terminal device 10 according to the present embodiment. As shown in FIG. 3, the terminal device 10 includes a microphone 101, a GPS 102, an acceleration sensor 103, a clock 104, a touch panel 105, a CPU 111, a ROM 112, a RAM 113, a task DB 121, a feature quantity DB 122, an exercise feature quantity DB 123, a specific character string uttered time DB 124, a speaker 131, a display 132, and a communication I/F 141.

### (1) Input Unit

As shown in FIG. 3, the terminal device 10 includes the microphone 101, the global positioning system (GPS) 102, the acceleration sensor 103, the clock 104, and the touch panel 105. These structural elements can be regarded as an input unit to which information is to be input. Besides them, the input unit may include any structural elements such as a camera, gyro sensor, biosensor, button, and keyboard, for example. The input unit has the function of inputting user-related information which will be described later.

The microphone 101 collects ambient sound. For example, the microphone 101 collects voice of the user or ambient sound. The microphone 101 may have a microphone amplifier circuit that subjects an audio signal obtained through the microphone to amplification processing, an analog to digital (A/D) converter, and a signal processing circuit that subjects audio data to processing such as noise removal and sound source separation or the like.

The GPS 102 detects positional information of the terminal device 10. The GPS 102 receives a GPS signal from a GPS satellite, for example, to detect positional information including the latitude, longitude, and altitude of the device, and outputs the detected positional information. Note that, instead of or along with the GPS 102, the terminal device 10 may have a device that detects positional information using any other technology. For example, instead of or along with the GPS 102, the terminal device 10 may have a device that detects positional information by means of Wi-Fi (registered trademark), transmission/reception with a mobile phone, PHS, smartphone, or the like, near field communication, or the like.

The acceleration sensor 103 detects the acceleration of the terminal device 10. The acceleration sensor 103 detects the acceleration by means of any system such as an optical system or a semiconductor system. The number of axes for which the acceleration is to be detected is arbitrary, and may be three axes, for example.

The clock 104 detects time information. The clock 104 detects time information by means of any system of quartz type or electric wave type.

The touch panel 105 detects a touch operation made by the user. Typically, the touch panel 105 is configured integrally with the display 132 which will be described later, and detects a touch operation on an image displayed on the display 132.

### Control Unit

As shown in FIG. 3, the terminal device 10 includes the central processing unit (CPU) 111, the read only memory (ROM) 112, and the random access memory (RAM) 113. These structural elements can be regarded as a control unit that controls the overall operation within the terminal device 10. The control unit may include any other structural elements besides them. The control unit has the function of controlling each structural element included in the terminal device 10, and, has the function of particularly processing user-related information. Note that each structural element included in the terminal device 10 shall operate on the basis of control exerted by the control unit, and description concerning this will be omitted below. For example, that the control unit controls the output unit so as to output information will be simply described as that the terminal device 10 outputs information.

The CPU 111 functions as an arithmetic processing device and a control device, and controls the overall operation within the terminal device 10 in accordance with various programs. The terminal device 10 may be implemented by a microprocessor or the like instead of or along with the CPU 111, and more briefly, may be implemented by an electronic circuit. The ROM 112 stores programs, operation parameters, and the like to be used. The RAM 113 temporarily stores parameters varying as appropriate and the like. Here, a flow of processing in the CPU 111 will be described in more detail with reference to FIG. 4.

FIG. 4 is a diagram for describing a flow of processing in the CPU 111 of the terminal device 10 according to the present embodiment. As shown in FIG. 4, processing performed by the CPU 111 includes processing performed by an acquisition unit 151, a learning unit 152, and an output control unit 153. Since the operations of these structural elements will be described later in detail, brief description will be given here. The acquisition unit 151 acquires user-related information for output to the learning unit 152 and the output control unit 153. The learning unit 152 generates and outputs normal state information indicating the normal state of the user on the basis of the user-related information. The normal state information is typically a feature quantity obtained from user-related information, and is stored by the feature quantity DB 122 which will be described later. The output control unit 153 generates and outputs output information on the basis of the user-related information, the normal state information stored in the feature quantity DB 122, and task information stored in the task DB 121. The output information may include any type of data such as image data, text data, and sound data. The output information is output by the speaker 131 or the display 132 which will be described later, or is transmitted by the communication I/F 141 which will be described later.

### Storage Unit

As shown in FIG. 3, the terminal device 10 includes a task data base (DB) 121 and the feature quantity DB 122. These structural elements can be regarded as a storage unit that temporarily or permanently stores information utilized by the terminal device 10. Besides them, the storage unit may include a DB for storing any type of information. The storage unit has the function of storing normal state information and task information.

The task DB 121 stores tasks to be provided for the user. The feature quantity DB 122 stores a feature quantity typically obtained from user-related information as the normal state information. The feature quantity DB 122 includes a DB that stores any feature quantities, such as the exercise feature quantity DB 123 that stores exercise feature quantities and the specific character string uttered time DB 124 that stores a specific character string uttered time which is a time at which a specific character string is uttered.

### Output Unit

As shown in FIG. 3, the terminal device 10 includes the speaker 131 and the display 132. These structural elements can be regarded as an output unit that outputs information. Besides them, the output unit may include any structural elements such as a vibration device and a lamp. The output unit has the function of outputting output information.

The speaker 131 outputs sound. The speaker 131 may have a digital to analog (D/A) converter and an amplifier, and via them, converts sound data into an analog signal for output (that is, playback).

Display 132 The display 132 outputs an image (still image/moving image). The display 132 is implemented by, for example, a liquid crystal display (LCD), an organic light-emitting diode (OLED), or the like.

### Communication Unit

As shown in FIG. 3, the terminal device 10 includes the communication I/F 141. The communication I/F 141 can be regarded as a communication unit for transmitting/receiving information. The communication unit has the function of communicating with the terminal device 30 or the server 60.

The communication I/F 141 is a communication module for transmitting/receiving information to/from another device in a wired/wireless manner. The communication I/F 141 makes communication by means of a communication system such as, for example, LAN, wireless LAN, Wi-Fi, Bluetooth, or LTE.

### 3. Technical Characteristics

A configuration example of the terminal device 10 has been described above. Subsequently, technical characteristics of the terminal device 10 will be described.

### 3.1. User-Related Information

The terminal device 10 (for example, the acquisition unit 151) acquires user-related information concerning the user. The user-related information includes at least any of information obtained by sensing the user, such as user’s biological information, activity information indicating an activity, and voice information, for example. Besides, the user-related information may include information concerning the family (particularly, parents) of the user. In addition, the user-related information may include information indicating a task fulfillment status which will be described later.

### 3.2. Prior Learning

The terminal device 10 (for example, the learning unit 152) leans the normal state of the user on the basis of the user-related information. For example, the terminal device 10 learns a feature quantity extracted on the basis of the user-related information as the normal state of the user. Besides, the terminal device 10 may learn a chronological change in feature quantity as the normal state. The latter example will be described later in detail. A learning result (for example, an extracted feature quantity or a chronological change in feature quantity) is stored in the feature quantity DB 122 as normal state information. The normal state to be learned can be considered in various manners. An example thereof will be described below in detail. Note that timing (in more detail, a time or period) at which the user-related information to be used for learning is acquired will be referred to as a first time.

### Normal State Concerning Exercise

For example, the terminal device 10 learns the normal state at the first time concerning exercise of the user. More specifically, the terminal device 10 calculates a feature quantity (hereinafter also referred to as an exercise feature quantity) concerning the exercise capacity (or physical capacity, physical fitness) of the user on the basis of acceleration information, positional information, or information indicating an activity of the user such as voice. As an example, learning of the normal state concerning acceleration information will be described with reference to FIG. 5.

FIG. 5 is a flowchart showing an example of a flow of prior learning processing performed by the terminal device 10 according to the present embodiment. As shown in FIG. 5, the terminal device 10 first acquires time information and acceleration information (step S102). Next, the terminal device 10 calculates a feature quantity (step S104). Then, the terminal device 10 records the calculated feature quantity in the exercise feature quantity DB 123 (step S106).

The exercise feature quantity can be considered in various manners. For example, the exercise feature quantity may include information indicating an instantaneous force. The information indicating an instantaneous force may be expressed as, for example, the maximum value of the absolute value of acceleration in a unit time. The terminal device 10 records information indicating an instantaneous force per unit time in the exercise feature quantity DB 123 in association with time information. An example of an instantaneous force table of the exercise feature quantity DB 123 is shown in Table 1 below.

TABLE 1

| Time (JST) | Instantaneous force |
|---|---|
| 2016/10/1 0:00 | 0 |
| 2016/10/1 1:00 | 0 |
| 2016/10/1 2:00 | 0 |
| 2016/10/1 3:00 | 0 |
| 2016/10/1 4:00 | 0 |
| 2016/10/1 5:00 | 0 |
| 2016/10/1 6:00 | 1 |
| 2016/10/1 7:00 | 2 |
| 2016/10/1 8:00 | 3 |
| 2016/10/1 9:00 | 3 |
| 2016/10/1 10:00 | 3 |
| 2016/10/1 11:00 | 3 |
| 2016/10/1 12:00 | 2 |
| 2016/10/1 13:00 | 2 |
| 2016/10/1 14:00 | 1 |
| 2016/10/1 15:00 | 1 |
| 2016/10/1 16:00 | 2 |
| 2016/10/1 17:00 | 2 |
| 2016/10/1 18:00 | 2 |
| 2016/10/1 19:00 | 2 |
| 2016/10/1 20:00 | 1 |
| 2016/10/1 21:00 | 0 |
| 2016/10/1 22:00 | 0 |
| 2016/10/1 23:00 | 0 |

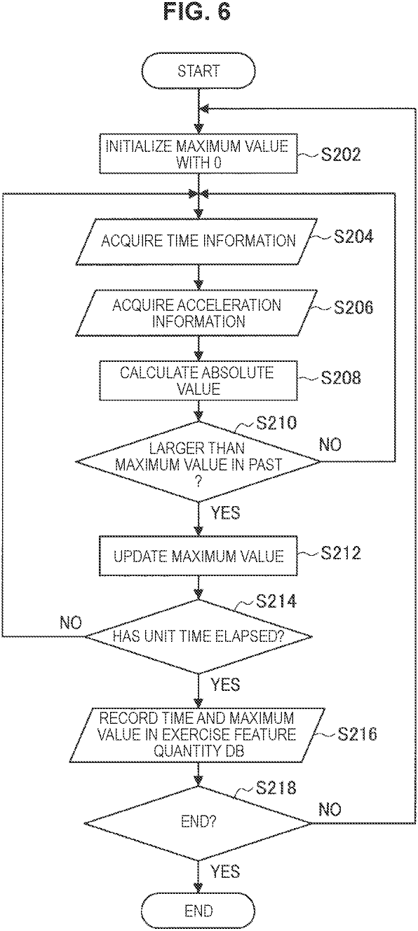

A flow of processing for learning such a table will be described below with reference to FIG. 6.

FIG. 6 is a flowchart showing an example of a flow of prior learning processing performed by the terminal device 10 according to the present embodiment. As shown in FIG. 6, the terminal device 10 first initializes the maximum value with 0 (step S202). Next, the terminal device 10 acquires time information (step S204), and acquires acceleration information (step S206). Next, the terminal device 10 calculates the absolute value of acceleration (step S208). Next, the terminal device 10 determines whether or not the calculated absolute value is larger than the maximum value in the past (step S210), and in the case where it is equal to or smaller, returns to step S204 again (step S210/NO), and in the case where it is larger, updates the maximum value (step S210/YES, S212). Next, the terminal device 10 determines whether or not the unit time has elapsed (step S214), and in the case where the unit time has not elapsed, returns to step S204 again (step S214/NO), and in the case where the unit time has elapsed, records the time and the maximum value in the exercise feature quantity DB 123 (step S214/YES, S216). Next, the terminal device 10 determines whether or not to terminate the processing (step S218), and in the case where the processing is not to be terminated, returns to step S202 again (step S218/NO), and in the case where the processing is to be terminated, terminates the processing (step S218/YES).

The feature quantity concerning an instantaneous force has been described above.

Besides, the exercise feature quantity may include information indicating the amount of activities. The information indicating the amount of activities may be expressed as, for example, an integrated value of the absolute value of acceleration per unit time. The terminal device 10 records information indicating the amount of activities per unit time in the exercise feature quantity DB 123 in association with time information. An example of an amount-of-activities table of the exercise feature quantity DB 123 is shown in Table 2 below.

TABLE 2

| Time (JST) | Amount of activities |
|---|---|
| 2016/10/1 0:00 | 0 |
| 2016/10/1 1:00 | 0 |
| 2016/10/1 2:00 | 0 |
| 2016/10/1 3:00 | 0 |
| 2016/10/1 4:00 | 0 |
| 2016/10/1 5:00 | 0 |
| 2016/10/1 6:00 | 10 |
| 2016/10/1 7:00 | 50 |
| 2016/10/1 8:00 | 120 |
| 2016/10/1 9:00 | 150 |
| 2016/10/1 10:00 | 150 |
| 2016/10/1 11:00 | 150 |
| 2016/10/1 12:00 | 100 |
| 2016/10/1 13:00 | 100 |
| 2016/10/1 14:00 | 20 |
| 2016/10/1 15:00 | 10 |
| 2016/10/1 16:00 | 50 |
| 2016/10/1 17:00 | 80 |
| 2016/10/1 18:00 | 50 |
| 2016/10/1 19:00 | 50 |
| 2016/10/1 20:00 | 10 |
| 2016/10/1 21:00 | 0 |
| 2016/10/1 22:00 | 0 |
| 2016/10/1 23:00 | 0 |

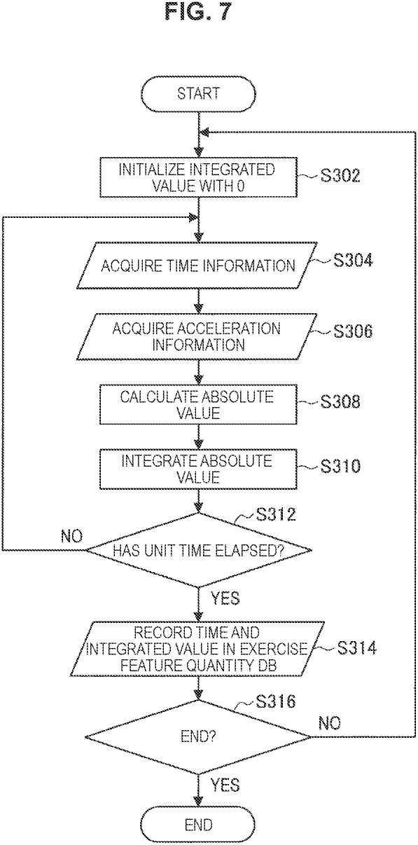

A flow of processing for learning such a table will be described below with reference to FIG. 7.

FIG. 7 is a flowchart showing an example of a flow of prior learning processing performed by the terminal device 10 according to the present embodiment. As shown in FIG. 7, the terminal device 10 first initializes the integrated value with 0 (step S302). Next, the terminal device 10 acquires time information (step S304), and acquires acceleration information (step S306). Next, the terminal device 10 calculates the absolute value of acceleration (step S308). Next, the terminal device 10 integrates the calculated absolute value into the integrated value (step S310). Next, the terminal device 10 determines whether or not the unit time has elapsed (step S312), and in the case where the unit time has not elapsed, returns to step S304 again (step S312/NO), and in the case where the unit time has elapsed, records the time and the integrated value in the exercise feature quantity DB 123 (step S312/YES, S314). Next, the terminal device 10 determines whether or not to terminate the processing (step S316), and in the case where the processing is not to be terminated, returns to step S302 again (step S316/NO), and in the case where the processing is to be terminated, terminates the processing (step S316/YES).

### Normal State Concerning Voice

For example, the terminal device 10 learns the normal state at the first time concerning voice of the user. More specifically, the terminal device 10 calculates a feature quantity concerning memory of the user indicated by voice of the user. As a feature quantity to be calculated, an utterance frequency of a specific character string uttered naturally when the user has forgotten something, for example, can be considered. An example of such a specific character string is shown in Table 3 below.

TABLE 3

Specific character string
what
that
Let me see
What was that
Ummm
I can’t remember
though I remembered

For example, the terminal device 10 records time information (for example, an uttered time) at which the specific character string is detected in the specific character string uttered time DB 124. An example of a table of the specific character string uttered time DB 124 is shown in Table 4 below.

TABLE 4

Time (JST)
2016/10/1 6:10
2016/10/1 10:15
2016/10/1 16:35
2016/10/1 18:00

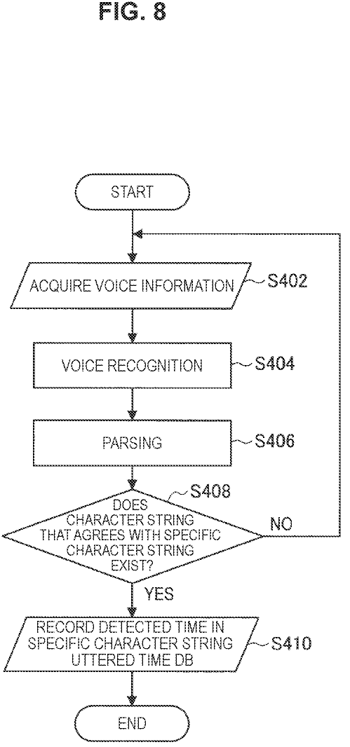

A flow of processing for learning such a table will be described below with reference to FIG. 8.

FIG. 8 is a flowchart showing an example of a flow of prior learning processing performed by the terminal device 10 according to the present embodiment. First, the terminal device 10 acquires voice information (step S402). Next, the terminal device 10 performs voice recognition (step S404), and further performs parsing (step S406). Next, the terminal device 10 determines whether or not a character string that agrees with the specific character string exists (step S408), and in the case where the character string does not exist, returns to step S402 again (step S408/NO), and in the case where the character string exists, records the detected time in the specific character string uttered time DB 124 (step S410).

### Others

Besides, the terminal device 10 may learn the normal state on the basis of various types of information.

For example, the terminal device 10 may learn the normal state at the first time on the basis of an interaction between a virtual pet that the user cares for and the user. More specifically, the terminal device 10 may calculate feature quantities concerning memory and attention of the user indicated by the cared status of the virtual pet. As the feature quantities to be calculated, whether or not the virtual pet is cared for at a scheduled time, a difference between the scheduled time and a time when caring is actually performed, and the like are considered, for example.

### 3.3. Task Provision

The terminal device 10 (for example, the output control unit 153) detects an abnormal state of the user by referring the acquired user-related information to the learned normal state. Then, when the abnormal state is detected, the terminal device 10 provides a task for suppressing the detected abnormal state. In more detail, the terminal device 10 compares the normal state at the first time in the past and the normal state at the second time based on user-related information at a second time (for example, at present) later than the first time to detect progress to abnormality higher than or equal to a predetermined level from the normal state at the first time as an abnormal state. Then, the output control unit 153 provides a task for suppressing the detected progress. The progress to abnormality here refers to progress of a decline due to aging, for example. In addition, the predetermined level refers to a level (for example, the average value for the same age, or the like) at which progress of a decline due to aging is acceptable, for example. That is, the abnormal state in the present specification refers to progress of a decline due to aging detected by comparing the normal state in the past and the current normal state, rather than merely referring to that user-related information indicates an abnormal value at certain timing. The terminal device 10 is capable of detecting progress of a decline due to aging and providing a task for suppressing the detected progress of a decline. Then, the user can suppress progress of a decline due to aging by fulfilling the provided task. In addition, as compared with the present situation in which a decline in physical capacity and intellectual capacity that progresses with aging cannot be evaluated without considerable costs caused by carrying out a physical fitness measurement and memory test performed by a specialist, and the like, the terminal device 10 is capable of evaluating a decline due to aging more briefly. Note that providing a task refers to requesting the user to perform some activities (for example, a movement, utterance, and the like).

### Abnormal State Detection Stage

processing of detecting an abnormal state will be described below. as described above, the terminal device 10 compares the normal state at the first time and the normal state at the second time to detect progress of a decline due to aging. as an example, an example in the case where the normal state at the first time and the normal state at the second time are exercise feature quantities such as instantaneous forces will be described below.

### Case of Exercise Feature Quantity

For example, assume that the second time is “2017/10/1”, and “2016/10/1” of the same day of the previous year shown in Table 1 above is the first time. An example of an instantaneous force table of the exercise feature quantity DB 123 recorded for the second time is shown in Table 5 below.

TABLE 5

| Time (JST) | Instantaneous force |
|---|---|
| 2017/10/1 0:00 | 0 |
| 2017/10/1 1:00 | 0 |
| 2017/10/1 2:00 | 0 |
| 2017/10/1 3:00 | 0 |
| 2017/10/1 4:00 | 0 |
| 2017/10/1 5:00 | 0 |
| 2017/10/1 6:00 | 0 |
| 2017/10/1 7:00 | 1 |
| 2017/10/1 8:00 | 2 |
| 2017/10/1 9:00 | 3 |
| 2017/10/1 10:00 | 3 |
| 2017/10/1 11:00 | 3 |
| 2017/10/1 12:00 | 2 |
| 2017/10/1 13:00 | 2 |
| 2017/10/1 14:00 | 1 |
| 2017/10/1 15:00 | 1 |
| 2017/10/1 16:00 | 2 |
| 2017/10/1 17:00 | 2 |
| 2016/10/1 18:00 | 1 |
| 2016/10/1 19:00 | 1 |
| 2016/10/1 20:00 | 0 |
| 2016/10/1 21:00 | 0 |
| 2016/10/1 22:00 | 0 |
| 2016/10/1 23:00 | 0 |

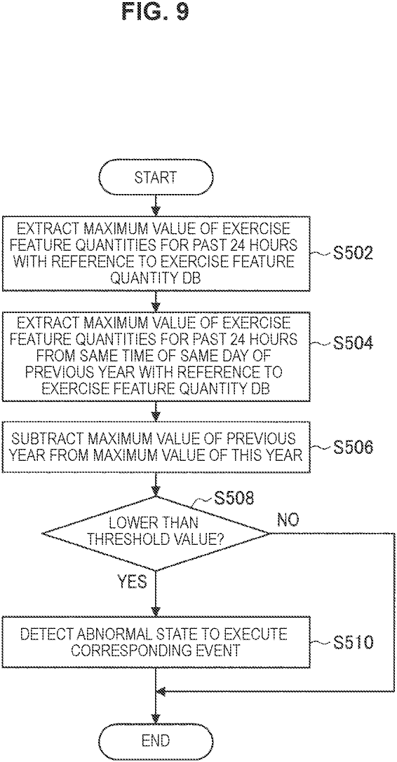

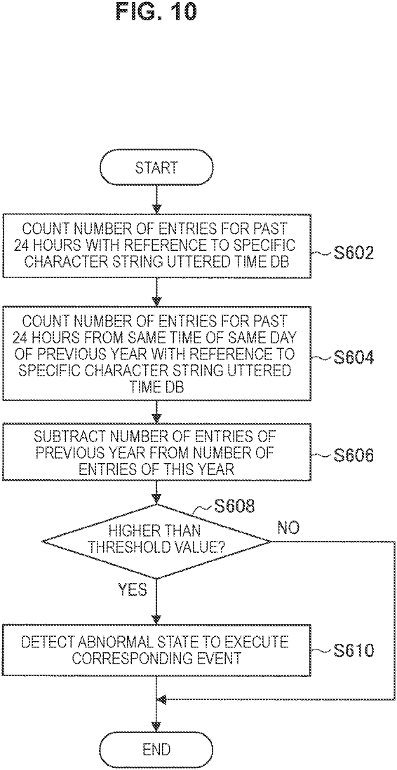

Comparing Table 1 for the first time and Table 5 for the second time, the instantaneous force is lower in Table 5 than in Table 1 at “6:00”, “7:00”, “8:00”, “18:00”, “19:00”, and “20:00”. In the case where the amount of decrease exceeds a threshold value, the terminal device 10 detects a decline in physical fitness of the user as an abnormal state. Note that the example in which the normal state at the first time is the normal state learned on the same day of the previous year is shown here, whilst the present technology is not limited to such an example. The interval between the first time and the second time is arbitrary, and as the normal state at the first time to be compared with, a state obtained by performing statistical processing such as averaging the normal states before and after that time may be used. Hereinafter, a flow of processing for detecting the abnormal state concerning an exercise feature quantity such as an instantaneous force will be described with reference to FIG. 9.

FIG. 9 is a flowchart showing an example of a flow of processing of detecting an abnormal state performed by the terminal device 10 according to the present embodiment. As shown in FIG. 9, the terminal device 10 first extracts the maximum value of exercise feature quantities for the past 24 hours with reference to the exercise feature quantity DB 123 (step S502). Next, the terminal device 10 extracts the maximum value of exercise feature quantities for the past 24 hours from the same time of the same day of the previous year with reference to the exercise feature quantity DB 123 (step S504). Next, the terminal device 10 calculates a subtraction value obtained by subtracting the maximum value of the previous year from the maximum value of this year (step S506). Next, the terminal device 10 determines whether or not the subtraction value is lower than a threshold value (step S508), and in the case where it is determined that the subtraction value is lower, detects an abnormal state to execute a corresponding event (step S508/YES, S510), and in the case where it is determined that the subtraction value is not lower, directly terminates the processing.

Note that executing a corresponding event typically refers to providing a task for suppressing an abnormal state. Besides, a notification to a third party which will be described later or the like may be performed.

### Case of Specific Character String Uttered Time

The example of detecting an abnormal state concerning an exercise feature quantity has been described above. Subsequently, as an example, an example of detecting an abnormal state concerning a specific character string uttered time will be described below.

For example, assume that the second time is “2017/10/1”, and the first time is “2016/10/1” of the same day of the previous year shown in Table 4 above. An example of a table of the specific character string uttered time DB 124 recorded for the second time is shown in Table 6 below.

TABLE 6

Time(JST)
2017/10/1 7:00
2017/10/1 7:05
2017/10/1 8:10
2017/10/1 8:30
2017/10/1 8:45
2017/10/1 8:50
2017/10/1 12:50
2017/10/1 13:00
2017/10/1 15:40
2017/10/1 16:00

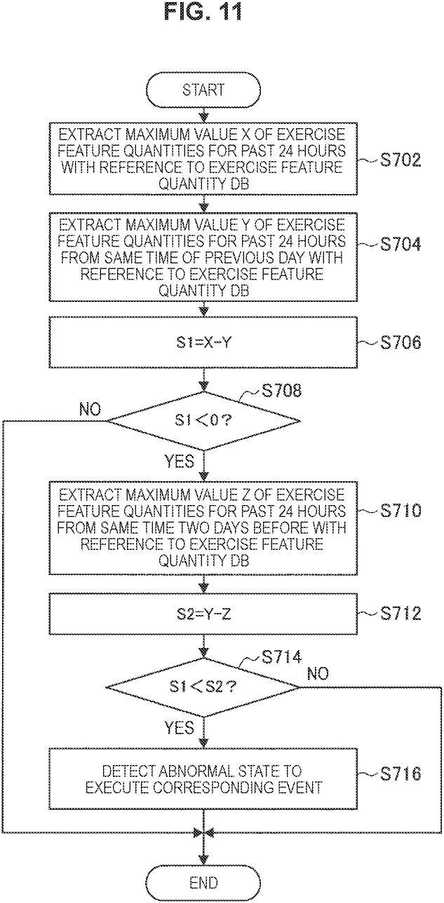

Comparing Table 4 for the first time and Table 6 for the second time, the number of entries (that is, the number of utterance of a specific character string) is larger in Table 6 than in Table 4. In the case where the amount of increase exceeds a threshold value, the terminal device 10 detects a decline in memory of the user as an abnormal state. A flow of processing for detecting an abnormal state concerning a specific character string uttered time will be described below with reference to FIG. 10.

FIG. 10 is a flowchart showing an example of a flow of processing of detecting an abnormal state performed by the terminal device 10 according to the present embodiment. As shown in FIG. 10, the terminal device 10 first counts the number of entries for the past 24 hours with reference to the specific character string uttered time DB 124 (step S602). Next, the terminal device 10 counts the number of entries for the past 24 hours from the same time of the same day of the previous year with reference to the specific character string uttered time DB 124 (step S604). Next, the terminal device 10 calculates a subtraction value obtained by subtracting the number of entries of the previous year from the number of entries of this year (step S606). Next, the terminal device 10 determines whether or not the subtraction value is higher than a threshold value (step S608), and in the case where it is determined that the subtraction value is higher, detects an abnormal state to execute a corresponding event (step S608/YES, S610), and in the case where it is determined that the subtraction value is not higher, directly terminates the processing.

### Case of Chronological Change in Feature Quantity

An example in the case where the normal state at the first time and the normal state at the second time are feature quantities has been described above. Subsequently, an example in the case where the normal state at the first time and the normal state at the second time are chronological changes in feature quantity will be described.

For example, the terminal device 10 compares a chronological change in feature quantity calculated on the basis of the user-related information at the first time and a chronological change in feature quantity calculated on the basis of the user-related information at the second time to detect an abnormal state. A chronological change in feature quantity calculated on the basis of the user-related information may express the degree of progress of a decline due to aging. That is, the terminal device 10 compares the degree of progress of a decline due to aging at the first time and the degree of progress of a decline due to aging at the second time. Accordingly, the terminal device 10 becomes capable of detecting that the degree of progress of a decline due to aging has been accelerated, for example, as an abnormal state. In more detail, the terminal device 10 is capable of detecting that the degree of progress of a decline due to aging has increased nonlinearly as an abnormal state. Considering that it is generally difficult to completely eliminate a decline due to aging, it can be said that it is reasonable to detect that the degree of progress of a decline has been accelerated as an abnormal state.

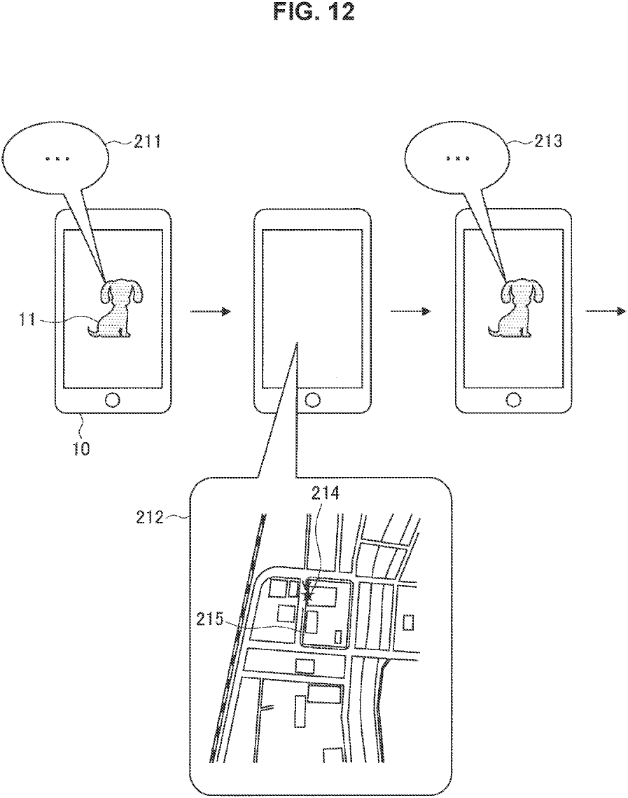

Hereinafter, as an example, an example in the case where the normal state at the first time and the normal state at the second time are chronological changes in exercise feature quantity such as an instantaneous force will be described with reference to FIG. 11. Note that FIG. 11 shows an example in which chronological changes in exercise feature quantity are calculated assuming that the second time is from a day before to the day, and the first time is from two days before to a day before.

FIG. 11 is a flowchart showing an example of a flow of processing of detecting an abnormal state performed by the terminal device 10 according to the present embodiment. As shown in FIG. 11, the terminal device 10 first extracts a maximum value X of exercise feature quantities for the past 24 hours with reference to the exercise feature quantity DB 123 (step S702). Next, the terminal device 10 extracts a maximum value Y of exercise feature quantities for the past 24 hours from the same time of the previous day with reference to the exercise feature quantity DB 123 (step S704). Next, the terminal device 10 subtracts the maximum value Y from the maximum value X to calculate a subtraction value S1 (step S706). Next, the terminal device 10 determines whether or not the subtraction value S1 is smaller than 0 (step S708). In the case where it is determined that the subtraction value S1 is not smaller than 0 (step S708/NO), the processing is terminated. In the case where it is determined that the subtraction value S1 is smaller than 0 (step S708/YES), the terminal device 10 extracts a maximum value Z of exercise feature quantities for the past 24 hours from the same time two days before with reference to the exercise feature quantity DB 123 (step S710). Next, the terminal device 10 subtracts the maximum value Z from the maximum value Y to calculate a subtraction value S2 (step S712). Next, the terminal device 10 determines whether or not the subtraction value S1 is smaller than the subtraction value S2 (step S714). In the case where it is determined that the subtraction value S1 is not smaller than the subtraction value S2 (step S714/NO), the processing is terminated. In the case where it is determined that the subtraction value S1 is smaller than the subtraction value S2 (step S714/YES), an abnormal state is detected and a corresponding event is executed (step S716).

### Supplement

In the foregoing, the abnormal state is a state detected quantitatively, such as a decrease in instantaneous force, a decrease in amount of activities, an increase in specific character string utterance frequency, and the like. Besides, the abnormal state may be a state detected qualitatively. That is, the terminal device 10 may detect an abnormal state on the basis of a qualitative change of the user. The qualitative change of the user refers to that the center of gravity has changed, an action protecting oneself has appeared, the user prefers to eat softer food, and the like, for example. Detection of such a qualitative change enables an abnormal state of the user to be detected more widely.

In addition, the terminal device 10 may detect an abnormal state on the basis of a task fulfillment status which will be described later. For example, an abnormal state may be detected on the basis of a decrease in the degree of task fulfillment (for example, the accuracy rate of a quiz) or the degree of decrease. In addition, the task is for suppressing progress of a decline due to aging, and besides, may be a concept including a mere event. For example, caring for the virtual pet may be for suppressing progress of a decline due to aging, or may be regarded as an event that occurs under a predetermined condition (for example, a scheduled time arrives, or the like).

In addition, the terminal device 10 may detect an abnormal state on the basis of information concerning the family of the user. For example, the terminal device 10 may set a criterion (for example, a threshold value) for determination for detecting an abnormal state on the basis of a tendency in physical fitness of the parents of the user, a tendency in a decline in physical fitness, or the like. Considering genetics, setting based on such family information enables an abnormal state of the user to be detected more appropriately.

### Task Providing Stage

Processing of providing a task will be described below. As described above, the terminal device 10 provides a task for, in the case where progress of a decline due to aging is detected, suppressing the progress of a decline due to aging.

For example, when progress of a decline in physical fitness of the user is detected as an abnormal state on the basis of the normal state of the user concerning exercise, the terminal device 10 provides a task for suppressing the progress of a decline in physical fitness. Specifically, in the case where an abnormal state is detected by the processing described above with reference to FIG. 9 or FIG. 11, for example, the terminal device 10 provides a task for suppressing the progress of a decline in physical fitness. The user can suppress the progress of a decline in physical fitness due to aging by fulfilling the provided task.

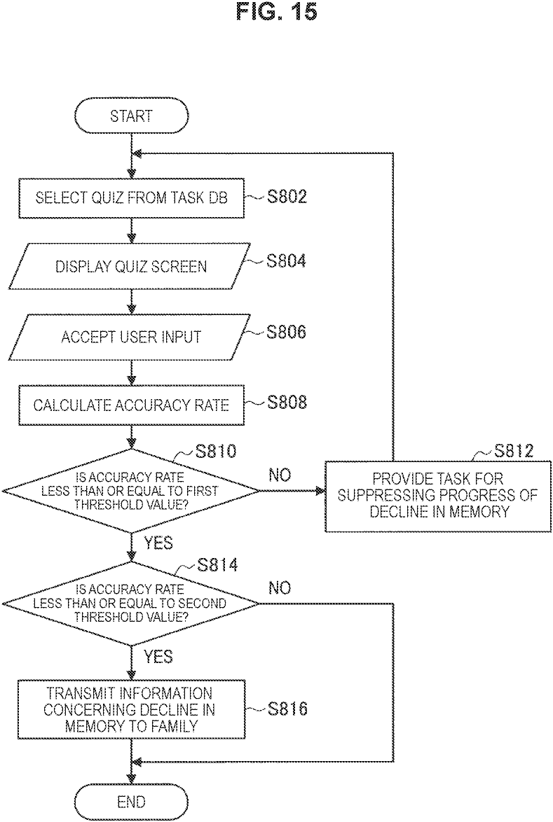

For example, when progress of a decline in memory of the user is detected as the abnormal state on the basis of the normal state concerning voice of the user, the terminal device 10 provides a task for suppressing the progress of a decline in memory. Specifically, in the case where an abnormal state is detected by the processing described above with reference to FIG. 10, for example, the terminal device 10 provides a task for suppressing the progress of a decline in memory. In addition, for example, when progress of a decline in memory of the user on the basis of the normal state based on an interaction between the virtual pet that the user cares for and the user is detected as an abnormal state, the terminal device 10 provides a task for suppressing the progress of a decline in memory. Specifically, in the case where an abnormal state is detected on the basis of a task fulfillment status which will be described later with reference to FIG. 14 or FIG. 16, for example, the terminal device 10 provides a task for suppressing the progress of a decline in memory. The user can suppress the progress of a decline in memory due to aging by fulfilling the provided task.

Here, the terminal device 10 controls the virtual pet that the user cares for so as to provide a task for the user. Accordingly, the user can be provided with a task through an interaction with the virtual pet that the user usually cares for. Since it is considered that, in the case where a task is provided by an attached virtual pet, the user can fulfill the task while having fun, it is possible to effectively suppress progress of a decline due to aging.

### Contents of Task

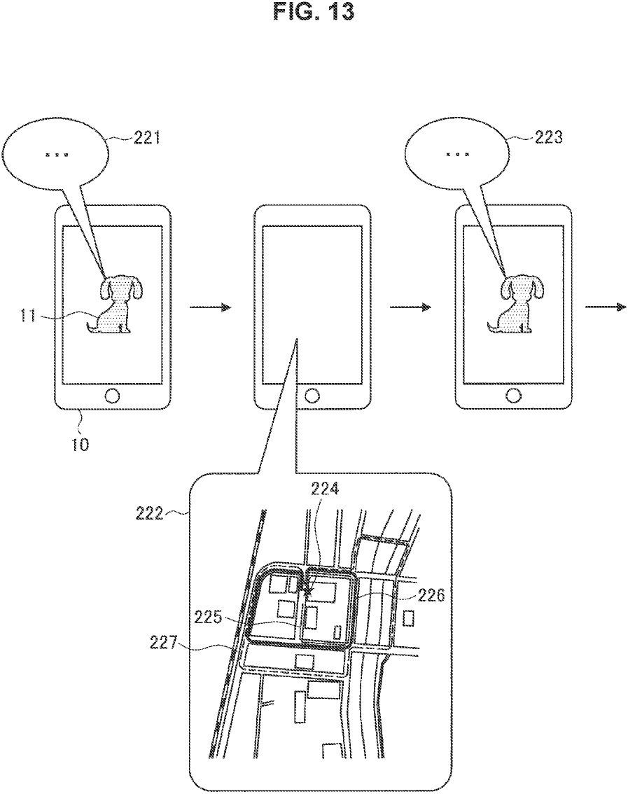

Contents of a task will be specifically described below. First, an example of a task of walking will be described with reference to FIG. 12.

FIG. 12 is a diagram for describing an example of a user interface (UI) for a task provided by the terminal device 10 according to the present embodiment. As shown in FIG. 12, the virtual pet 11 operating on the terminal device 10 outputs by sound a message 211 for notifying that the task of walking has been provided for the user. The message 211 is “Let’s go for a walk”, for example. Next, the terminal device 10 displays information 212 indicating a walking course to notify the user of the walking course. Then, the virtual pet 11 outputs by sound a message 213 for notifying the user of the start of walking, and starts navigation of the walking course. The message 213 is “It’s a 10-minute course. I will guide you”, for example. The information 212 indicating the walking course will be described in detail. The information 212 indicating the walking course includes map information about the surroundings of a current location 214 of the user and a walking course 215. The walking course 215 is a course that is optimum (that is, imposes a necessary and sufficient load) for the current physical fitness of the user, for example. Note that a course search may be performed in the background.

The task may be a task that does not allow direct presumption that it is a task for suppressing an abnormal state. From another point of view, the task may be a task implicitly indicating that it is a task for suppressing an abnormal state. Accordingly, since the user is not directly notified that a decline due to aging is progressing, the user can fulfill the task without receiving a shock. Further, since it is considered that the sense of duty that the user feels for fulfilling the task is reduced, the user can fulfill the task while having fun, and it is possible to effectively suppress progress of a decline due to aging.

For example, the task may be a task that suggests an action with a heavier load than an action that the user usually performs. Since, for the user, the task that suggests an action with a heavier load than a usual action is to perform an action for achieving the same object as usual merely by another method, for example, it is possible to naturally suppress progress of a decline with aging.

More specifically, the task may be a task that suggests a moving path with a heavier exercise load than a moving path that the user usually uses. For example, assume the case where the user usually takes a walk. In that case, the terminal device 10 provides a task that suggests a walking course with a heavier exercise load, such as longer in distance or steeper, than a usual walking course. With such a task, it is considered that progress of a decline in physical fitness can be suppressed. Besides, various tasks are considered. For example, a task that suggests an action using fingertips more than usual may be provided. With such a task, it is considered that progress of a decline in memory can be suppressed. For example, for example, a quiz task for suppressing progress of a decline in memory which will be described later with reference to FIG. 14 and a feeding task which will be described later with reference to FIG. 16 are considered. In addition, a task of singing a new song for suppressing progress of a decline in memory and language ability, a conversation task through use of words more difficult than usual, and the like are also considered.

In addition, the terminal device 10 may control a task load in accordance with the degree of progress of a decline due to aging. For example, the terminal device 10 provides a low-load task in the case where the degree of progress of a decline is low, and in the case where the degree of progress of a decline is high, provides a high-load task. Such control enables a task with an appropriate load to be provided in accordance with the degree of progress of a decline, and it is possible to efficiently suppress the progress of a decline.

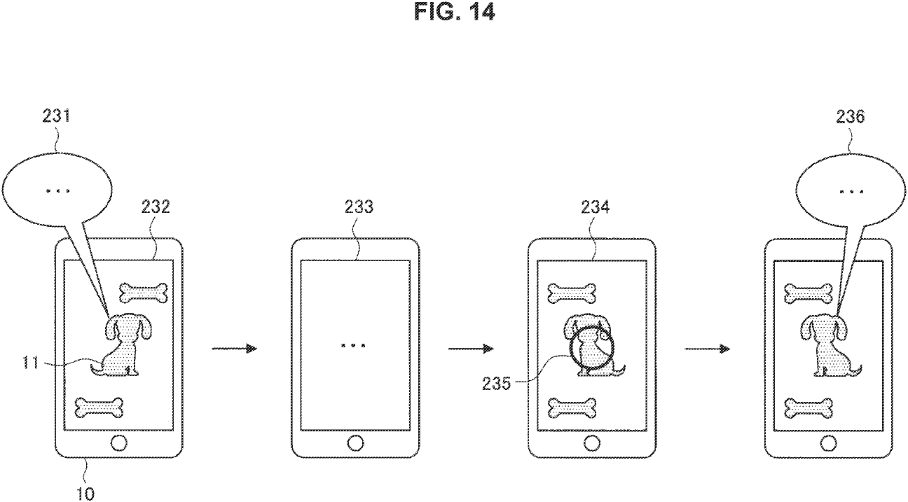

A task that suggests a walking course with an exercise load higher than a usual walking course and a load in accordance with the degree of progress of a decline will be specifically described below with reference to FIG. 13.

FIG. 13 is a diagram for describing an example of UI for a task provided by the terminal device 10 according to the present embodiment. As shown in FIG. 13, the virtual pet 11 operating on the terminal device 10 outputs by sound a message 221 for notifying that a task of walking has been provided for the user. The message 221 is “Let’s go for a walk”, for example. Next, the terminal device 10 displays information 222 indicating a walking course to notify the user of the walking course. Then, the virtual pet 11 outputs by sound a message 223 for notifying the user of the start of walking, and starts navigation of the walking course. The message 223 is “It’s a 10-minute course. I will guide you”, for examples. The information 222 indicating the walking course will be described in detail. The information 222 indicating the walking course includes map information about the surroundings of a current location 224 of the user and a walking course 225, a walking course 226 with a high load, and a walking course 227 with a still high load. In FIG. 13, the walking courses 225, 226, and 227 are displayed at the same time, but may be displayed selectively. The walking course 225 is a course that is optimum (that is, imposes a necessary and sufficient load) for the current physical fitness of the user, for example. The walking course 226 is a walking course with a higher load than the walking course 225, for example, and is selected in the case where slight progress of a decline in exercise capacity is detected. The walking course 227 is a walking course with a still higher load than the walking course 226, for example, and is selected in the case where progress of a significant decline in exercise capacity is detected. For example, the walking course 227 is selected in the case where a decrease in exercise capacity twice that of the case where the walking course 226 is selected is detected (that is, in the example shown in FIG. 11, S1/S2≈2), and may have a distance twice that of the walking course 226. Note that a course search may be performed in the background.

Note that the example where a walking course is presented in advance has been shown above, whilst the present technology is not limited to such an example. For example, stopping on the way while walking on the usual walking course 225 may be proposed. In addition, a task to be provided may be changed in accordance with a user’s situation, such as in the case where the user has forgotten to go to the toilet, a walking course by way of the toilet is proposed, for example.

The contents of a task to be provided is stored in the task DB 121 as task information. The terminal device 10 provides a task on the basis of task information stored in the task DB 121. The terminal device 10 may have an artificial intelligence function, and may provide a unique task. In addition, the terminal device 10 may provide a task on the basis of task information obtained by inquiring a specialized institution via the server 60, for example.

### Another Event

Besides provision of a task in the case where an abnormal state is detected, another event corresponding to the abnormal state may be executed.

### Control Over Method of Detecting Abnormal State

For example, the terminal device 10 may control a method of detecting an abnormal state in accordance with a result of detection of an abnormal state. Specifically, the terminal device 10 may control the interval between the first time and the second time in accordance with the degree of progress of a decline due to aging. For example, by shortening the interval between the first time and the second time in the case where the degree of progress is high, it is possible to detect abrupt progress of a decline.

### Control Over Task Provision Frequency

For example, the terminal device 10 may control a task providing method in accordance with a result of detection of an abnormal state. Specifically, the terminal device 10 may control a task provision frequency in accordance with the degree of progress of a decline due to aging. For example, by increasing the provision frequency in the case where the degree of degree of progress is high, it is possible to suppress progress of a decline more strongly.

### Notification to Third Party

For example, the terminal device 10 may make a notification to a third party. Relatively gentle progress of a decline is suppressed by provision of a task, while, by early notifying a third party (typically, family) of an abrupt decline, it is possible to have the third party assist in suppression of progress of the decline. In addition, it is also possible to share the manner in which the user leads everyday life with the third party.

### First Notification

For example, the terminal device 10 may transmit information concerning a user’s fulfillment status of a provided task to the third party. For example, in the case where the degree of task fulfillment has decreased, the terminal device 10 transmits information indicating that to the family (that is, parents) of the user. The information may be transmitted by e-mail or short message service (SMS), for example. Accordingly, it is possible to early notify the family of information to be worried about, such as abrupt progress of a decline.

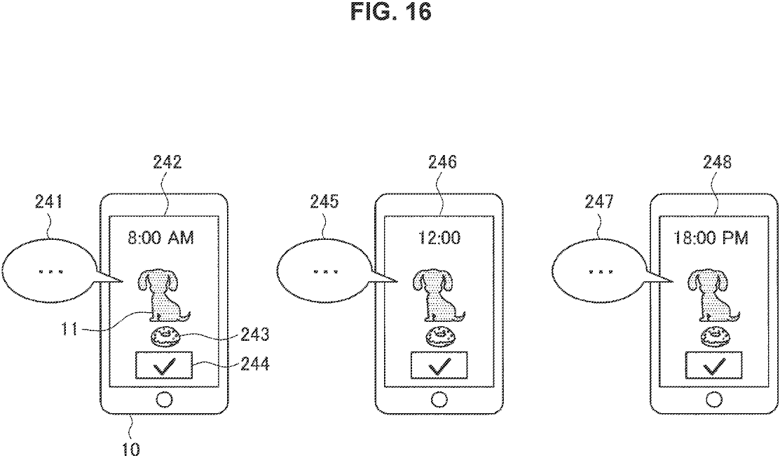

For example, as a task for suppressing progress of a decline in memory, a quiz task may be assumed. An example of a quiz task will be described below with reference to FIG. 14, and then, processing of notifying the third party of information concerning a quiz task fulfillment status will be described with reference to FIG. 15.

FIG. 14 is a diagram for describing an example of UI for a task provided by the terminal device 10 according to the present embodiment. As shown in FIG. 14, the virtual pet 11 operating on the terminal device 10 outputs by sound a message 231 for notifying that a quiz task has been provided for the user, and the terminal device 10 displays a screen 232 including the virtual pet 11. The message 231 is “I’ll give you a quiz”, for example. Next, the terminal device 10 displays a screen 233 including a description of the quiz, and describes the contents of the quiz to the user. The screen 233 including a description of the quiz includes text such as “Touch a portion having changed from before on the next screen”, for example. Next, the terminal device 10 may display a quiz screen 234 to accept a reply from the user. For example, the user touches a region 235 he/she considers that a change has been made from the screen 232. Then, the virtual pet 11 outputs by sound a message 236 indicating a result of evaluation of the reply from the user. The message 236 is “Close!”, for example.

FIG. 15 is a flowchart showing an example of a flow of processing of notifying a third party performed by the terminal device 10 according to the present embodiment. As shown in FIG. 15, the terminal device 10 first selects a quiz task from the task DB 121 (step S802). Next, the terminal device 10 displays a quiz screen (step S804), and accepts input of a reply from the user (step S806). Next, the terminal device 10 calculates the accuracy rate (step S808), and determines whether or not the accuracy rate is less than or equal to a first threshold value (step S810). In the case where it is determined that the accuracy rate is not less than or equal to the first threshold value (step S810/NO), the terminal device 10 provides a task for suppressing progress of a decline in memory (step S812). As such a task, a different quiz task may be selected, for example. On the other hand, in the case where it is determined that the accuracy rate is less than or equal to the first threshold value (step S810/YES), the terminal device 10 determines whether or not the accuracy rate is less than or equal to a second threshold value (step S814). The second threshold value is a threshold value lower than the first threshold value, and that the accuracy rate is lower than the second threshold value indicates that a decline in memory should be worried about, for example. In the case where it is determined that the accuracy rate is not less than or equal to the second threshold value (step S814/NO), the processing is directly terminated. On the other hand, in the case where it is determined that the accuracy rate is less than or equal to the second threshold value (step S814/YES), the terminal device 10 transmits information concerning a decline in memory to the family (step S816).

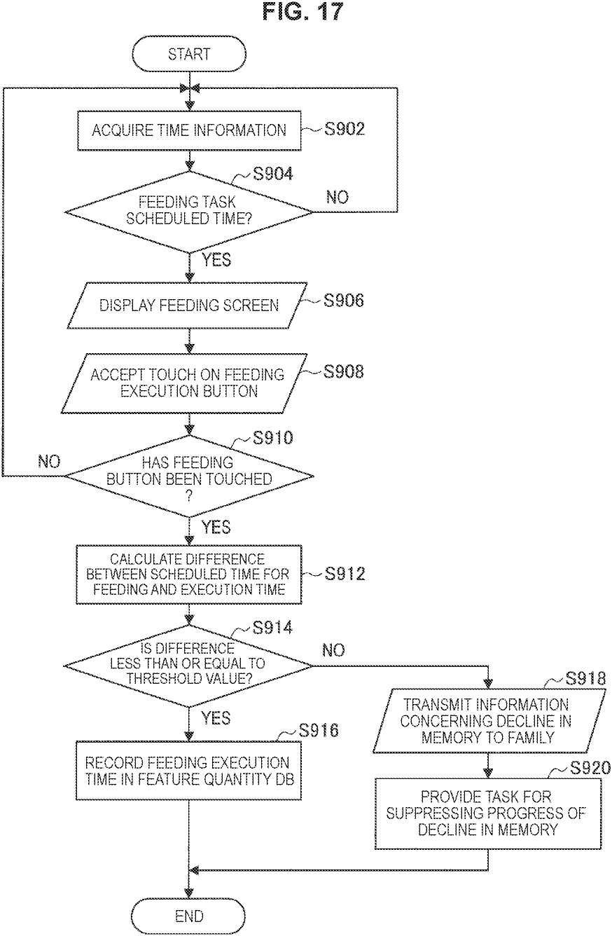

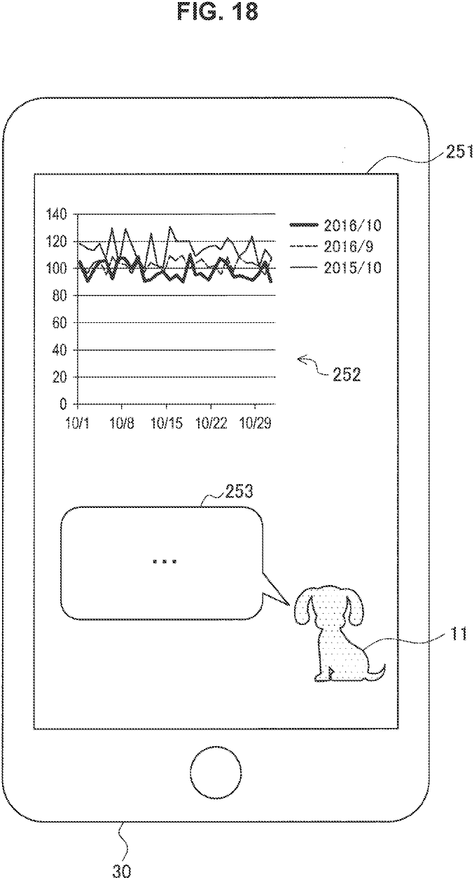

In addition, as a task for suppressing progress of a decline in memory, a task of caring for the virtual pet may also be assumed. An example of a task of caring for the virtual pet will be described below with reference to FIG. 16, and then, processing of notifying the third party of information concerning the fulfillment status of the task of caring for the virtual pet will be described with reference to FIG. 17.

FIG. 16 is a diagram for describing an example of UI for a task provided by the terminal device 10 according to the present embodiment. As shown in FIG. 16, at breakfast, the virtual pet 11 operating on the terminal device 10 outputs by sound a message 241 for notifying that a feeding task has been provided for the user, and the terminal device 10 displays a feeding screen 242. The message 241 is “Give me breakfast!”, for example. Time information, the virtual pet 11, a food icon 243, and a feeding execution button 244 are included on the feeding screen 242, and when the user touches the feeding execution button 244, fulfillment of the feeding task is completed. Next, at lunch, the virtual pet 11 outputs by sound a message 245 for notifying that a feeding task has been provided for the user, and the terminal device 10 displays a feeding screen 246. The message 245 is “Give me lunch!”, for example. The feeding screen 246 is similar to the feeding screen 242 except for the change in time, and when the user touches the feeding execution button, fulfillment of the feeding task is completed. Next, at dinner, the virtual pet 11 outputs by sound a message 247 for notifying that a feeding task has been provided for the user, and the terminal device 10 displays a feeding screen 248. The message 247 is “Give me dinner!”, for example. The feeding screen 248 is similar to the feeding screen 242 except for the change in time, and when the user touches the feeding execution button, fulfillment of the feeding task is completed.

FIG. 17 is a flowchart showing an example of a flow of processing of notifying a third party performed by the terminal device 10 according to the present embodiment. As shown in FIG. 17, the terminal device 10 first acquires time information (step S902), and determines whether or not it is a scheduled time for a feeding task (step S904). In the case where it is determined that it is not the scheduled time (step S904/NO), the processing returns to step S902 again. On the other hand, in the case where it is determined that it is the scheduled time (step S904/YES), the terminal device 10 displays a feeding screen (step S906), and accepts a touch on the feeding execution button (step S908). Next, the terminal device 10 determines whether or not the feeding execution button has been touched (step S910). In the case where it is determined that the feeding execution button has not been touched (step S910/NO), the processing returns to step S902 again. On the other hand, in the case where it is determined that the feeding execution button has been touched (step S910/YES), the terminal device 10 calculates the difference between the scheduled time for feeding and an execution time (step S912), and determines whether or not the difference is less than or equal to a threshold value (step S914). In the case where it is determined that the difference is less than or equal to the threshold value (step S914/YES), the terminal device 10 records a feeding execution time in the feature quantity DB 122 (step S916). On the other hand, in the case where it is determined that the difference is not less than or equal to the threshold value (step S914/NO), the terminal device 10 transmits information concerning a decline in memory to the family (step S918). Note that, instead of or along with this, the terminal device 10 may record the feeding execution time in the feature quantity DB 122. Then, the terminal device 10 provides a task for suppressing the progress of a decline in memory (step S920). Such a task may be a quiz task described with reference to FIG. 14, for example.

### Second Notification

For example, the terminal device 10 may transmit a summary concerning user-related information to the third party. For example, the terminal device 10 transmits, to the family of the user, information obtained by performing graphing, statistical processing, or addition of comments on feature quantities such as an instantaneous force and the amount of activities, calculated on the basis of the user-related information. Accordingly, it is possible to share the manner in which the user leads everyday life, such as usual activities or habits, with the family. An example of information to be notified will be described with reference to FIG. 18.

FIG. 18 is a diagram for describing an example of information of which the third party is to be notified by the terminal device 10 according to the present embodiment. As shown in FIG. 18, a screen 251 including a summary concerning user-related information reported by the virtual pet 11 of the user is displayed on the terminal device 30 of the family. A graph 251 and a message 253 concerning the user-related information are included on the screen 251. The graph 251 represents transitions of the amount of activities for one month on “2016/10”, “2016/9” which is one month before, and “2015/10” which is the same month of the previous year, targeted for summarizing. The message 253 is “The amount of activities has been reduced by about 7% relative to the same month last year. The amount of activities has been reduced by about 1% relative to the last month. Try to go out more” concerning the amount of activities of the user, for example. Besides, as a summary concerning user-related information, a message concerning memory of the user, such as “Mr. A says ‘What is that?’ frequently from around the last month. Maybe he is getting a little forgetful”, may be included. Here, “Mr. A” is the name of the user. In addition, as a summary concerning user-related information, a message concerning memory of the user, such as “Mr. A forgets to feed me about once in two days. He often forgets dinner particularly. But he feeds me when I remind him, so he seems to remember me”, may be included.

Note that the terminal device 10 may have an artificial intelligence function, and may create the above-described summary independently. Besides, the terminal device 10 may transmit user-related information to cause the server 60 to create the summary, or cause a specialized institution to create the summary.

### Supplement

Besides, for example, a third party may be notified that the exercise capacity or the like of the user has been improved. In addition, the user himself/herself may be notified of the above-described information.

### 4. Conclusion

An embodiment of the present disclosure has been described above in detail with reference to FIG. 1 to FIG. 18. As described above, the information processing system 1 according to the present embodiment acquires user-related information concerning the user, learns the normal state of the user on the basis of the user-related information, and when an abnormal state of the user is detected by referring the acquired user-related information to the learned normal state, provides a task for suppressing the detected abnormal state. By evaluation of the user-related information based on the normal state, more specifically, by comparison between the normal state at the first time and the normal state at the second time, long-term progress of a decline is detected as an abnormal state. Since a task for suppressing this long-term progress of a decline is provided, the user can suppress the long-term progress of a decline by fulfilling the task.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, in the above-described embodiment, the example where a task for suppressing progress of a decline due to aging is provided has been mainly described, whilst the present technology is not limited to such an example. For example, in the case where a decline progresses in a long term because of a disease, lack of exercise, or the like, a task for suppressing that progress may be provided. That is, the present technology is widely useful for health maintenance. Further, a task for not only suppressing a decrease in capacity, but also improving capacity, may be provided.

Each device described in the present specification may be achieved as an individual device, or a part or the whole may be achieved as separate devices. For example, in the functional configuration example of the terminal device 10 shown in FIG. 3 and FIG. 4, the task DB 121 and the feature quantity DB 122, as well as the acquisition unit 151, the learning unit 152, and the output control unit 153 may be included in the server 60. In that case, the terminal device 10 transmits the user-related information to the server 60, and the server 60 performs learning, task selection, UI creation, and the like on the basis of the user-related information, and transmits the results to the terminal device 10 to cause the terminal device 10 to output the results. That is, the above-described functions provided by the information processing system 1 according to the present embodiment may be provided by cooperation of a plurality of devices included in the information processing system 1. Conversely, the above-described functions provided by the information processing system 1 according to the present embodiment may be provided by the terminal device 10 alone.

Note that a series of processing performed by the respective devices described in the present specification may be achieved using any of software, hardware, and a combination of software and hardware. Programs that configure software are stored in advance in recording media (non-transitory media) provided inside or outside the respective devices, for example. Then, each program is read into RAM when execution by a computer, for example, and is executed by a processor such as CPU.

In addition, processing described in the present specification using flowcharts and sequence diagrams may not be necessarily executed in the illustrated order. Some processing steps may be executed in parallel. In addition, additional processing steps may be adopted, or some processing steps may be omitted.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.
(1) An information processing system including:
   an acquisition unit configured to acquire user-related information concerning a user;
   a learning unit configured to learn a normal state of the user on the basis of the user-related information; and
   an output control unit configured to, when an abnormal state of the user is detected by referring the acquired user-related information to the learned normal state, exert control so as to provide a task for suppressing the detected abnormal state.
(2) The information processing system according to (1), in which
   the output control unit compares the normal state at a first time and the normal state at a second time based on the user-related information at the second time later than the first time to detect progress to abnormality higher than or equal to a predetermined level from the normal state at the first time as the abnormal state, and exerts control so as to provide the task for suppressing the progress.
(3) The information processing system according to (2), in which
   the output control unit compares a chronological change in feature quantity calculated on the basis of the user-related information at the first time and a chronological change in feature quantity calculated on the basis of the user-related information at the second time to detect the abnormal state.
(4) The information processing system according to (2) or (3), in which
   the output control unit detects the abnormal state on the basis of a qualitative change of the user.
(5) The information processing system according to any one of (2) to (4), in which
   the learning unit learns the normal state concerning exercise of the user, and
   when progress of a decline in physical fitness of the user is detected as the abnormal state on the basis of the normal state concerning exercise of the user, the output control unit exerts control so as to provide the task for suppressing the progress of a decline in physical fitness.
(6) The information processing system according to any one of (2) to (5), in which
   the learning unit learns the normal state concerning voice of the user, and
   when progress of a decline in memory of the user is detected as the abnormal state on the basis of the normal state concerning voice of the user, the output control unit exerts control so as to provide the task for suppressing the progress of a decline in memory.
(7)The information processing system according to any one of (2) to (6), in which
   the learning unit learns the normal state on the basis of an interaction between a virtual organism that the user cares for and the user, and
   when progress of a decline in memory of the user is detected as the abnormal state on the basis of the normal state based on the interaction between the virtual organism that the user cares for and the user, the output control unit exerts control so as to provide the task for suppressing the progress of a decline in memory.
(8) The information processing system according to any one of (2) to (7), in which
   the output control unit controls a load of the task in accordance with a degree of the progress.
(9) The information processing system according to any one of (2) to (8), in which
   the output control unit controls an interval between the first time and the second time in accordance with a degree of the progress.
(10) The information processing system according to any one of (2) to (9), in which
   the output control unit controls a provision frequency of the task in accordance with a degree of the progress.
(11) The information processing system according to any one of (1) to (10), in which
   the task is a task that does not allow direct presumption that it is a task for suppressing the abnormal state.
(12) The information processing system according to (11), in which
   the task is a task that suggests an action with a heavier load than an action that the user usually performs.
(13) The information processing system according to (12), in which
   the task is a task that suggests a moving path with a heavier exercise load than a moving path that the user usually uses.
(14) The information processing system according to any one of (1) to (13), in which
   the output control unit controls a virtual organism that the user cares for so as to provide the task for the user.
(15) The information processing system according to any one of (1) to (14), further including:
   a communication unit, in which
   the output control unit controls the communication unit so as to transmit information concerning a fulfillment status of the provided task by the user to a third party.
(16) The information processing system according to any one of (1) to (15), further including:
   a communication unit, in which
   the output control unit controls the communication unit so as to transmit a summary concerning the user-related information to a third party.
(17) A recording medium having recorded thereon a program for causing a computer to function as:
   an acquisition unit configured to acquire user-related information concerning a user;
   a learning unit configured to learn a normal state of the user on the basis of the user-related information; and
   an output control unit configured to, when an abnormal state of the user is detected by referring the acquired user-related information to the learned normal state, exert control so as to provide a task for suppressing the detected abnormal state.
(18) An information processing method including:
   acquiring user-related information concerning a user;
   learning a normal state of the user on the basis of the user-related information; and
   when an abnormal state of the user is detected by referring the acquired user-related information to the learned normal state, exerting control by a processor so as to provide a task for suppressing the detected abnormal state.

REFERENCE SIGNS LIST

| | |
|---|---|
| 1 | information processing system |
| 10 | terminal device |
| 11 | virtual pet |
| 20 | user |
| 30 | terminal device |
| 40 | family |
| 50 | network |
| 60 | server |
| 101 | microphone |
| 102 | GPS |
| 103 | acceleration sensor |
| 104 | clock |
| 105 | touch panel |
| 111 | CPU |
| 112 | ROM |
| 113 | RAM |
| 121 | task DB |
| 122 | feature quantity DB |
| 123 | exercise feature quantity DB |
| 124 | specific character string uttered time DB |
| 131 | speaker |
| 132 | display |
| 141 | communication I/F |
| 151 | acquisition unit |
| 152 | learning unit |
| 153 | output control unit |

1. An information processing system comprising:
an acquisition unit configured to acquire user-related information concerning a user;
a learning unit configured to learn a normal state of the user on a basis of the user-related information; and
an output control unit configured to, when an abnormal state of the user is detected by referring the acquired user-related information to the learned normal state, exert control so as to provide a task for suppressing the detected abnormal state.

2. The information processing system according to claim 1, wherein
the output control unit compares the normal state at a first time and the normal state at a second time based on the user-related information at the second time later than the first time to detect progress to abnormality higher than or equal to a predetermined level from the normal state at the first time as the abnormal state, and exerts control so as to provide the task for suppressing the progress.

3. 3 The information processing system according to claim 2, wherein
the output control unit compares a chronological change in feature quantity calculated on a basis of the user-related information at the first time and a chronological change in feature quantity calculated on a basis of the user-related information at the second time to detect the abnormal state.

4. The information processing system according to claim 2, wherein
the output control unit detects the abnormal state on a basis of a qualitative change of the user.

5. The information processing system according to claim 2, wherein
the learning unit learns the normal state concerning exercise of the user, and
when progress of a decline in physical fitness of the user is detected as the abnormal state on a basis of the normal state concerning exercise of the user, the output control unit exerts control so as to provide the task for suppressing the progress of a decline in physical fitness.

6. The information processing system according to claim 2, wherein
the learning unit learns the normal state concerning voice of the user, and
when progress of a decline in memory of the user is detected as the abnormal state on a basis of the normal state concerning voice of the user, the output control unit exerts control so as to provide the task for suppressing the progress of a decline in memory.

7. The information processing system according to claim 2, wherein
the learning unit learns the normal state on a basis of an interaction between a virtual organism that the user cares for and the user, and
when progress of a decline in memory of the user is detected as the abnormal state on a basis of the normal state based on the interaction between the virtual organism that the user cares for and the user, the output control unit exerts control so as to provide the task for suppressing the progress of a decline in memory.

8. claim 8 The information processing system according to claim 2, wherein
the output control unit controls a load of the task in accordance with a degree of the progress.

9. claim 9 The information processing system according to claim 2, wherein
the output control unit controls an interval between the first time and the second time in accordance with a degree of the progress.

10. The information processing system according to claim 2, wherein
the output control unit controls a provision frequency of the task in accordance with a degree of the progress.

11. The information processing system according to claim 1, wherein
the task is a task that does not allow direct presumption that it is a task for suppressing the abnormal state.

12. The information processing system according to claim 11, wherein
the task is a task that suggests an action with a heavier load than an action that the user usually performs.

13. The information processing system according to claim 12, wherein
the task is a task that suggests a moving path with a heavier exercise load than a moving path that the user usually uses.

14. The information processing system according to claim 1, wherein
the output control unit controls a virtual organism that the user cares for so as to provide the task for the user.

15. The information processing system according to claim 1, further comprising:
a communication unit, wherein
the output control unit controls the communication unit so as to transmit information concerning a fulfillment status of the provided task by the user to a third party.

16. The information processing system according to claim 1, further comprising:
a communication unit, wherein
the output control unit controls the communication unit so as to transmit a summary concerning the user-related information to a third party.

17. A recording medium having recorded thereon a program for causing a computer to function as:
an acquisition unit configured to acquire user-related information concerning a user;
a learning unit configured to learn a normal state of the user on a basis of the user-related information; and
an output control unit configured to, when an abnormal state of the user is detected by referring the acquired user-related information to the learned normal state, exert control so as to provide a task for suppressing the detected abnormal state.

18. An information processing method comprising:
acquiring user-related information concerning a user;
learning a normal state of the user on a basis of the user-related information; and
when an abnormal state of the user is detected by referring the acquired user-related information to the learned normal state, exerting control by a processor so as to provide a task for suppressing the detected abnormal state.
